(19) United States
(12) Patent Application Publication
(10) Pub. No.: US 2016/0206232 A1
(43) Pub. Date: Jul. 21, 2016

(54) Methods and Apparatus for Optical Non-Invasive Blood Glucose Change Indication

(71) Applicant: Socrates Health Solutions, Inc., Dallas, TX (US)

(72) Inventor: Michael Bordelon, Sugar Land, TX (US)

(21) Appl. No.: 14/807,634

(22) Filed: Jul. 23, 2015

Related U.S. Application Data
(60) Provisional application No. 62/103,961, filed on Jan. 15, 2015.

(51) Int. Cl. A61B 5/1455; A61B 5/00; A61B 5/145

(52) U.S. Cl. CPC A61B 5/1455; A61B 5/14532; A61B 5/0017; A61B 5/742

(57) ABSTRACT

A method for performing an optical non-invasive blood glucose concentration change indication is disclosed, including: providing an optical energy source spaced from a photo-detector by a sensing area; transmitting energy from the optical energy source across human tissue disposed in the sensing area and onto the photo-detector; storing a first reading corresponding to a light intensity observed by the photo-detector; and displaying the first reading on a display of a user device, the first reading corresponding to a baseline blood glucose concentration. Apparatus for performing the method and additional embodiments are disclosed.

### RELATED APPLICATIONS

The present application claims priority to U.S. Provisional Application Ser. No. 62/103,961 (attorney docket SOC-1003P) filed on Jan. 15, 2015, entitled “Methods and Apparatus for Optical Non-Invasive Blood Glucose Change Indication,” which application is hereby incorporated in its entirety herein by reference.

### TECHNICAL FIELD

The embodiments relate generally to the use of optical non-invasive techniques to determine changes in blood glucose concentration.

### BACKGROUND

Blood glucose monitoring is of increasing interest and importance. Blood glucose monitoring is used, for example, by individuals with diabetes. Diabetes mellitus is a group of diseases causing abnormal blood sugar levels over a prolonged period of time. Diabetes is a result of either the pancreas not producing enough insulin, or the failure of the cells to respond to the insulin produced. As of November 2014, studies estimate that around 347 million people worldwide have the disease, Lancet, 378(9785):31-40 (2011).

Glucose monitors have improved from biochemical reactions in which a color change would be visually compared to a color chart, to electrochemical reactions in which a reaction with the glucose in the blood would be measured and read digitally. In the last 50 years, tests have gotten faster (from over a minute to just a few seconds) and easier (early tests required washing and blotting test strips), and lancets have evolved from steel strips with a point to spring-loaded needles. These changes have made home testing better, but the fact remains that drawing blood for testing using known glucose monitors is not only a potential health hazard, but also carries with it social stigma, pain, it produces medical waste that needs proper disposal, and the patient has to bear the cost of one time use test strips.

In addition to managing diabetes, interest in determining changes in blood glucose concentration is also increasing in healthy individuals. Uses of blood glucose information for athletic training, in dieting for weight loss, in determining proper food intake to support healthy exercise, and continuing increasing interest in improved nutrition and in certain modes of diet and the impact on the health of the individual (such as kosher, vegetarian, local sourced, low-carb, low-fat and other nutrition modes including paleo, gluten-free, and the like) all lead to additional and increasing interest in a capability to easily determine changes in blood glucose concentration.

One prior known approach to glucose concentration measurement is described in U.S. Pat. No. 8,743,355, (the '355 Patent), entitled “Simple Sugar Concentration Sensor and Method,” issued Jun. 3, 2014, which is hereby incorporated by reference herein in its entirety. The '355 Patent discloses optical sensing of the angular rotation of optical energy passed through a sample including a sugar, for example, glucose in a fluid such as blood. In particular the '355 Patent discloses using photosensitive detectors to sense the rotation of polarized light that passes through a sample, for example, through human tissue including blood.

The '355 Patent describes optical measurements made on a portion of the human ear using multiple polarizers to create polarized light, and a difference measurement taken between two photosensitive detectors, one with a polarizer, and one without. However, using the prior known approaches that are described in the '355 Patent, the readings obtained require additional accuracy and an increase in reproducibility in order to enable a practical glucose meter for individual and consumer or patient use.

The inventor of the present application has researched the approach of the '355 Patent and related non-invasive glucose measurements and has found that the prior known approaches described to date lack the accuracy, reproducibility in results, efficiency and ease of use needed to provide a practical commercial non-invasive glucose monitor.

Improvements are therefore needed in non-invasive glucose monitoring in order to address the deficiencies and the disadvantages of the prior known approaches. Solutions are needed that reduce the cost and complexity of the monitor system and which can accurately measure changes in blood glucose concentration.

### SUMMARY

Methods and apparatus for determining changes in blood glucose using optical non-invasive monitoring are provided. In the novel methods and apparatus, an arrangement has been unexpectedly discovered that uses only a single light source and a single photo-detector. Surprisingly, a measurement that correlates strongly to changes in blood glucose concentration is obtained using this novel approach.

In an aspect of the present application a method for performing an optical non-invasive blood glucose concentration change indication, comprising: providing an optical energy source spaced from a photo-detector by a sensing area; transmitting energy from the optical energy source across human tissue disposed in the sensing area and onto the photo-detector; storing a first reading corresponding to a light intensity observed by the photo-detector; and displaying the first reading on a display of a user device, the first reading corresponding to a baseline blood glucose concentration.

In another aspect of the present application, the above described method is performed and further comprising: subsequently, again transmitting energy from the optical energy source across the tissue disposed in the sensing area and onto the photo-detector; storing a second reading corresponding to the light intensity observed by the photo-detector; determining a difference between the first reading and the second reading; and displaying the difference between the first reading and the second reading indicating a change in blood glucose concentration from the baseline blood glucose concentration.

In yet another aspect of the present application, the above methods are performed and wherein transmitting energy from the optical energy source across human tissue disposed in the sensing area further comprises transmitting energy across a portion of a human ear.

In still another aspect of the present application, an apparatus includes: an illumination source; a photo-detector spaced from the illumination source by a sensing area configured for insertion of human tissue between the illumination source and the photo-detector; and circuitry coupled to the photo-detector for outputting readings corresponding to light intensity observed by the photo-detector for light transmitted by the illumination source.

In an additional aspect of the present application, the above described apparatus further includes: a transimpedance amplifier coupled to the photo-detector for receiving a current and outputting a voltage corresponding to light received by the photo-detector.

In yet another aspect of the present application, the above described apparatus further includes an analog to digital converter coupled to the transimpedance amplifier and outputting a digital signal corresponding to the voltage.

In still another aspect of the present application, the above described apparatus is provided and further comprises a microcontroller coupled to the analog to digital converter and coupled to the illumination source, and configured to control the illumination source and to store readings corresponding to the digital signal.

In yet another aspect of the present application, the above described apparatus is provided and further comprises a radio transceiver for transmitting data corresponding to the stored readings in the microcontroller.

Additional alternative arrangements are also described to form additional aspects of the present application.

### BRIEF DESCRIPTION OF THE DRAWINGS

For a more complete understanding of the illustrative embodiments described herein and the advantages thereof, reference is now made to the following descriptions taken in conjunction with the accompanying drawings, in which:

FIG. 1 depicts in a simple block diagram a non-invasive optical blood glucose concentration change system that forms an aspect of the present application;

FIG. 2 illustrates the use of the system of FIG. 1 in an example application for taking measurements applied to the tissue of the human ear;

FIG. 3 illustrates in a simplified block diagram a sensor apparatus that forms an additional aspect of the present application;

FIG. 4 illustrates in a circuit block diagram an example of circuitry arranged for use with the arrangements of the present application;

FIG. 5 illustrates in a simple block diagram the use of an example sensor of the arrangements of the present application in an application;

FIG. 6 illustrates in a simple block diagram a user device for use with the sensors of the arrangements of the present application;

FIG. 7 illustrates in a flowchart a method performed for taking readings using the sensors of the arrangements of the present application for use in determining changes in blood glucose concentration; and

FIG. 8 illustrates in a flowchart a method performed for operating a user device tin conjunction with the use of the sensors of the present application.

Corresponding numerals and symbols in the different figures generally refer to corresponding parts unless otherwise indicated. The figures are drawn to clearly illustrate the relevant aspects of the arrangements of the present application and are not necessarily drawn to scale.

### DETAILED DESCRIPTION

The making and using of example illustrative arrangements that form aspects of the present application are discussed in detail below. It should be appreciated, however, that the arrangements provide many applicable inventive concepts that can be embodied in a wide variety of specific contexts. The specific example arrangements discussed are merely illustrative of specific ways to make and use the various arrangements, and do not limit the scope of the specification, or the appended claims.

The inventor of the present application has surprisingly and unexpectedly discovered that a simple and effective method of determining changes in blood glucose concentration in an individual can be reliably and reproducibly obtained using a non-invasive optical measurement taken across human tissue such as at a portion of the human ear, for example. In sharp contrast to prior known approaches, the novel glucose change method requires only a single illumination source and a single photo-detector. The measurement is of light intensity changes. Unlike the known prior solutions, the novel measurements do not require advanced signal processing, spectroscopy, or the use of complex electronics. As a result a simple, affordable and effective battery powered appliance that can be worn continuously by a user can be utilized, and continuous monitoring of changes in the concentration of glucose in the blood can be readily attained. In some of the arrangements, a wearable sensor is linked by wire or linked by using a wireless interface to a user device. The user device can include portable devices such as tablet computers, smartphones, laptops, biometric sensor displays, fitness watches, or the like to provide a platform for a user interface. The user can select and change the display style, the sensitivity, and the frequency of the glucose readings for example. In addition, the blood glucose monitoring sensor can also be added to and the added features can provide additional biometric information such as temperature, pulse, pulse-oximeter, pedometer, GPS, accelerometers and the like. When the sensors are used together with a smartphone, smart watch or other portable user device, the sensors can provide an overall biometric sensing system. Because the sensor can be worn while the individual walks, runs, cycles or trains, and because the sensor can communicate to mobile user devices, a continuous monitor providing these biometric information can be formed including the novel blood glucose change indications.

While diabetic persons can use the blood glucose monitoring as information for managing their diabetes, many other applications are also contemplated for many individuals including both diabetic and healthy persons. Example applications of the arrangements of the present application include, but are not limited to, athletic training, nutrition, diet coaching and weight loss support, and the like.

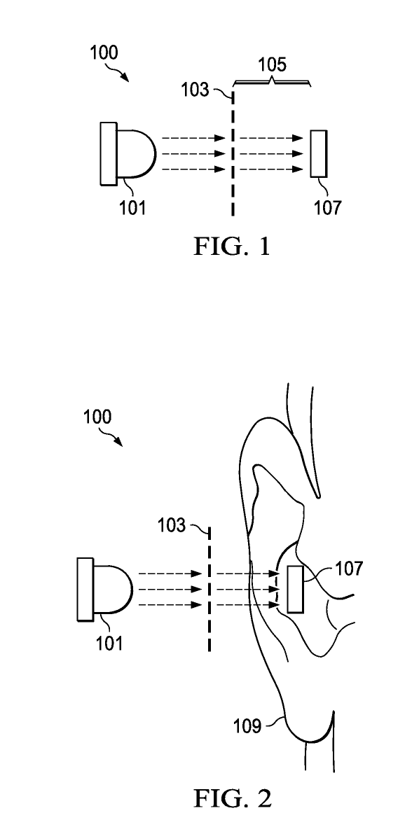

FIG. 1 depicts in a simple illustration the components of the optical non-invasive blood glucose change monitoring system 100. In FIG. 1, a source of optical energy such as an LED 101 is spaced from a photosensitive detector, or photodetector 107, by a sensing area 105. The system 100 will be used to transmit light across a portion of human tissue that is relatively thin, so the sensing area 105 can be a few millimeters wide. A polarizer 103 is shown between the optical source 101 and the sensing area. In one arrangement that has been implemented, a commercially available linear polarizer material was used. The use of the polarizer 103 is not required, and the fact this component is an optional feature is indicated by the dashed lines in FIG. 1.

FIG. 2 depicts system 100 shown in FIG. 1 while in use. In addition to the components shown above in FIG. 1, a human ear 109 is depicted. In the use of the sensor 100, the illumination source, component 101, is placed, in this non-limiting illustrative example, behind the fleshy part of the ear. The photodetector, component 107, is placed on the opposite side of a portion of the fleshy part of the ear, the pinna, so that light from the LED 101 traverses the tissue of the ear, and the light also is transmitted through any blood containing vessels within, before striking the photodetector 107. In an alternative arrangement that forms an additional aspect of the present application, the photodetector 107 can be behind the ear, and the LED 101 can be in front of the ear tissue, so long as the light traverses the tissue of the ear, the methods described below will operate. This alternative arrangement forms an additional aspect of the present application that is also contemplated by the inventor.

In a sensing operation, the LED 101 is illuminated briefly, and after allowing time for the system to stabilize, a reading corresponding to the light intensity received at the photodetector 107 is obtained. The LED 101 is then turned off. In additional and alternative embodiments contemplated by the inventor as providing further aspects of the present application, multiple readings can be taken in a short time, and the readings can be averaged or otherwise sampled. The use of multiple samples can be used to reduce noise errors or can be used to eliminate outlying samples or clearly erroneous results.

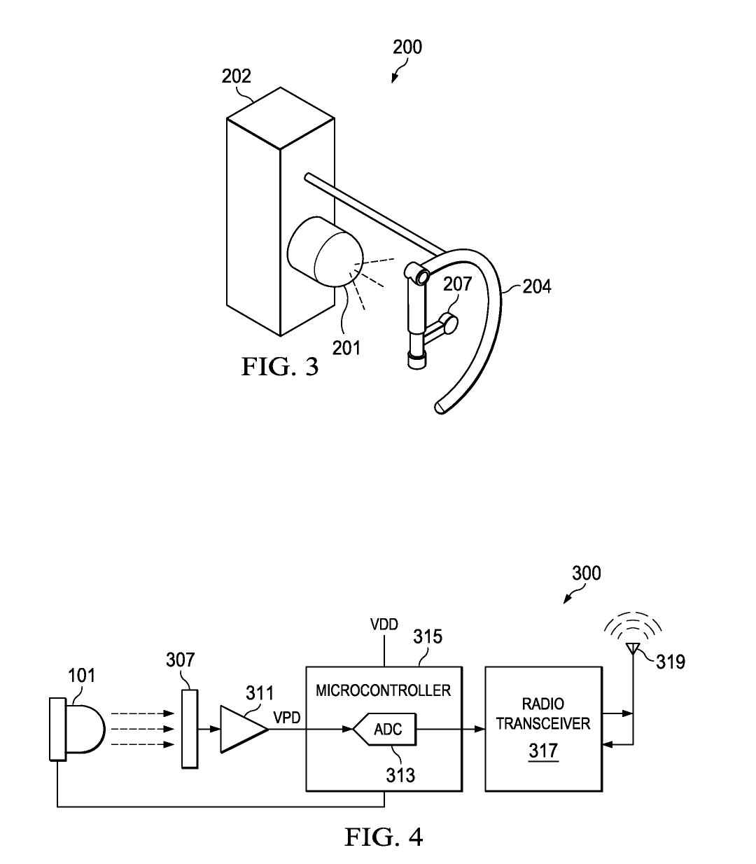

FIG. 3 illustrates, in a non-limiting example that is shown for the purpose of explanation and which does not limit the application and any appended claims, a novel sensor apparatus for optical non-invasive blood glucose change monitoring. The example sensor 200 is arranged to be comfortably worn on the human ear. However, in alternative arrangements, the sensor could have a different form and be of two or more pieces, for example, or otherwise be arranged. The various arrangements are configured with the common feature that a photodetector is arranged to sense light that is transmitted through human tissue containing blood. In this illustrative example, a portion of the sensor 200 including the photodetector 207 and a corresponding circuit board for electronics can be packaged in a comfortable shape for insertion into the outer portion of the ear canal, similar to the form factor of a hearing aid. The support 204 provides mechanical support between the chassis 202 and the photodetector module 207 and also provides a place to securely dispose an electrical connection, such as a wire, between the two portions 207 and 202. The LED 201 is mounted on the exterior of the chassis 202. Chassis 202 can also include a power source such as a watch battery or similar battery, which can be a disposable or rechargeable battery, and further chassis 202 can include communication electronics such as a Bluetooth or Wi-Fi transceiver integrated circuit and antenna as is further described below. Alternatively a simple wired connection can be used to receive output from sensor 200.

A transceiver device within module 202 can communicate to any user device such as a tablet, smartphone, a web browsing device, or a laptop or desktop computer. Component 201 represents an illumination source. In an example embodiment, a light emitting diode (LED) can be used as component 201 to produce optical energy in the form of signal light. In a non-limiting example, a red LED can be used as component 201. In another example, an alternate light source, such as a laser, can be used. Near-infrared light can also be used. In an example implementation, an LED of about 600-700 nanometer wavelength, such as a red LED, was used.

It has been surprisingly discovered by the inventor of the present application that the apparatus depicted in FIGS. 1-3 can provide reliable and robust indications of changes in blood glucose concentration. In one embodiment method, in operation the sensor 200 can be calibrated to a baseline glucose concentration. This can be done simply by taking a first reading that corresponds to the intensity of the light through the ear tissue, and the user can select that reading as the baseline. For example, the user can perform this action at any time when the user feels his or her glucose level is normal or stable. In one non-limiting example use, the user could make this initial reading in conjunction with use of a prior known glucose meter, for diabetes management, but this is not required nor necessary. Following this initial reading, which is now the baseline, additional readings using the sensor 200 can be made. These readings can be manually stimulated by the user, or in an alternative approach, the user can select a periodic reading interval, such as at 5 minute, 2 minute or other intervals. In any event the additional readings from the sensor are then displayed as a change in blood glucose concentration (as compared to the baseline value). The voltages corresponding to the light received by the photodetector for these additional readings can then be compared to the voltage recorded for the baseline reading and differences corresponding to changes in the light received can be displayed as changes in blood glucose concentration. The changes indicated can include increasing and decreasing changes.

The novel method described above was determined by making many trial readings using a device similar to the apparatus in FIGS. 1-3 for many test subjects. In these trials, a subject's blood glucose was first measured using both the novel apparatus such as is shown in FIGS. 1-3, and also independently, using a prior art glucose meter. A beverage intended to increase blood glucose concentration was then consumed by the subject. Following the consumption of the beverage the optical non-invasive sensor was used to take readings at selected time intervals for several hours. At these same time intervals a reading using the prior art glucose meter was also taken. The data provide a clear, incontrovertible and reproducible showing that changes in the readings corresponding to the light intensity transmitted through the ear tissue strongly correlate to the changes in the blood glucose concentration. The readings were independently confirmed using the prior art glucose meter. Using these results, it has been unexpectedly discovered that an optical non-invasive blood glucose change indication can be provided using a single photodiode and a single illumination source. In sharp contrast to the prior known solutions, spectroscopy techniques, or the need for measuring a very small optical rotation signal in the received light, and the corresponding need for performing other additional complex and extensive signal processing steps, are not required.

FIG. 4 depicts in a circuit block diagram a system 300 including the illumination source 301 and the photodetector 307 of the novel sensor, and further detailing certain functional circuit blocks used to output signals from the sensor. In system 300, an LED or other illumination source 301 is coupled to a micro-controller or micro-processor 315. When enabled by the micro-controller 315 the LED 301 will transmit light through the tissue. Photodetector 307 is positioned to receive the light transmitted through the tissue. The output of the photodetector 307 is coupled to a transimpedance amplifier 311. The output of the transimpedance amplifier 311 can be a voltage signal Vpd, for example, that corresponds to the light received at the photodetector 307 while the LED 301 was active.

The transimpedance amplifier 311 can be coupled to an analog to digital converter 313. In one example a micro-controller 315 supplied by Atmel Corporation, numbered the Atmega128 was used, this device includes a 10 bit analog to digital (ADC) converter such as 313 in FIG. 4 integrated with a programmable RISC processor. However, in implementing the circuitry shown in FIG. 4, a stand-alone ADC can also be used with any number of commercially available processors, micro-processors, or micro-controllers. Other possible implementations that can be used include creating application specific integrated circuits (ASICs), creating field programmable gate arrays (FPGAs), programming complex programmable logic devices (CPLDs), and the like. In the illustrative and non-limiting example of FIG. 4, the microcontroller 315 is further coupled to a radio transceiver device 317 that can transmit the data from the microcontroller 315 to a user device using antenna 319. In one illustrative example, a Bluetooth transceiver 317 can be used. In alternative embodiments other over the air interfaces can be used such as Wi-Fi. An antenna 319 is provided to enable sending and receiving of over the air data and control signals to and from the microcontroller 315. In additional alternative embodiments that are also contemplated as aspects of the present application, a wired interface can be used instead of the wireless or over the air interface. The wired connection or the wireless connection can be made to a user device such as a smartphone, tablet, laptop, notebook or desktop computer or web browser. Alternative arrangements include using a dedicated display unit and user interface device that can include an LED or LCD display, and that can further include user input buttons, for example.

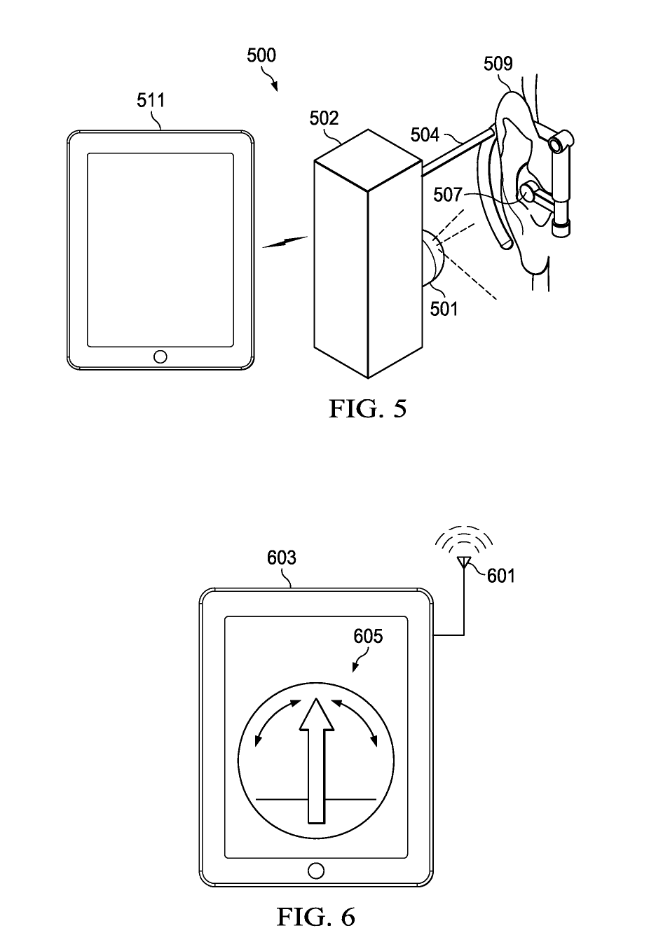

FIG. 5 illustrates in a simple diagram a system 500 and depicts the use of a wearable sensor 502 incorporating the features described above with a wireless connection to a user device 511. The user device 511 can be any one of a multitude of devices as described above, in an example implementation a smartphone was used. In FIG. 5, chassis 502 can include a battery and the microcontroller and transceiver devices as described above, and the LED 501 can be mounted on the exterior of the chassis 502. A photodetector 507 and associated electronics such as a transimpedance amplifier can be provided at one end of the support 504. In one example arrangement that is contemplated as an aspect of the present application, a wire is disposed on or within support 504 and this wire couples the transimpedance amplifier output to an analog to digital converter, as described above. The sensor 502 is configured be comfortably worn on the human ear with the LED 501 behind the pinna portion, and the photodetector 507 within the front side of the ear and in the outer portion of the ear canal, similar to a hearing aid form but not limited to that form.

In FIG. 6, a user device 603 is shown in operation with the sensors described above. An antenna 601 receives data from and sends signals to the sensor using an over the air interface. The interface can be implemented using Bluetooth, Wi-Fi, or other standard interfaces. Many user devices commercially available at this time include Bluetooth capability and transceiver devices are easily obtained to add Bluetooth capability to laptop computers, desktop computers, and the like if needed. A user interface application 605 is shown running on the user device 603. In this particular example arrangement, an indicator arrow is displayed and a baseline reading is shown at a centered point. Changes measured from the baseline are then indicated in an increasing or decreasing direction. While this arrangement for displaying the changes in blood glucose information has been implemented and is found to be useful, the user device can be programmed to present the information in any number of other manners and these variation are also contemplated by the inventor and fall within the scope of the present application. Bar charts, pie charts, vector graphs, data plots, thermometer plots, and the like can also be used. The user device can be a portable user device such as a tablet or smartphone. In an alternative arrangement that is also another aspect of the present application, a dedicated wireless or wired interface display and controller can be provided as a user device.

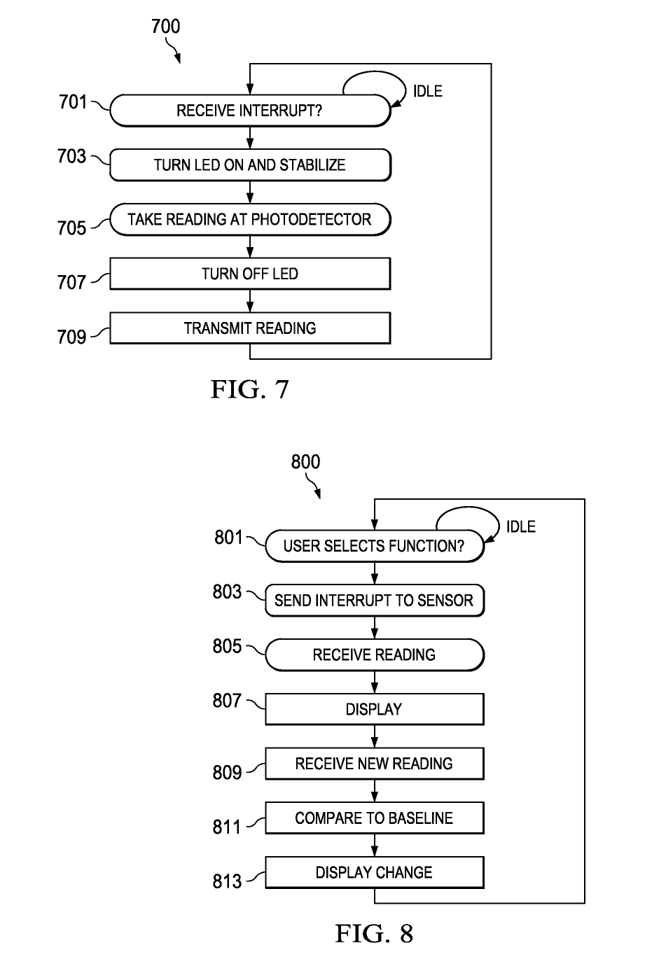

In FIG. 7, a method 700 that forms an additional aspect of the present application is shown in a flow diagram. At step 701, the method begins with a sensor in a power down or sleep mode. Using a deep sleep arrangement, the sensor can extend the battery life considerably by being inactive except for an occasional check to see if any new messages are being received over a wired or wireless interface, such as for example a Bluetooth interface. If an interrupt is received while in step 701, the method transitions to step 703. If no interrupt is received, the method remains in an Idle mode and returns to step 701.

At step 703, the method continues following an interrupt received on the communications interface. The microcontroller in the circuit as described above can turn on the LED and wait for a short time for the system components to stabilize. After the time elapses, which can be less than 1 second, the method continues to step 705. At step 705 a reading is taken at the photodetector. This reading can be obtained from a voltage output by a transimpedance amplifier coupled to the photodetector, for example, as described above.

At step 707 the LED is turned off. Turning off the LED as soon as the reading is taken reduces power consumption, extending battery life for the sensor.

At step 709, the reading is transmitted to the receiver over a wireless or wired interface. The method then returns to step 701 and awaits another interrupt signal on the interface.

While the steps and the illustration in FIG. 7 are arranged in an example order for purposes of illustration, the step order can be varied to form additional alternative arrangements that are also contemplated as part of the present application. For example, the steps 709 and 707 can be swapped or can be performed simultaneously. Also, although in this non-limiting example method interrupt messages are used to wake the circuitry, a timed wake up, or polling loop, or other method to begin the sensing can also be used, for example a simple user button on the body of the sensor could be used to begin the method. These variations are also contemplated as additional aspects of the present application.

FIG. 8 depicts in a method flow diagram, the steps of a method for operating an example user device with the sensors described above. At step 801, the user inputs a function selection that causes a reading to be needed. The user can select from a simple menu any command such as ‘start’, or ‘take a reading’, and cause the user interface program to begin. In one example arrangement that has been utilized, a smartphone application is used as the user interface program. However the arrangements contemplated by the inventor are not so limited, and can be varied. At step 803 the user device sends an interrupt to the sensor. As described with respect to FIG. 7 above, the method of operating the sensor can begin by receiving an interrupt over a wired or wireless interface such as a Bluetooth connection. At step 805, the user interface device receives a reading from the sensor. This occurs when the sensor method such as shown in FIG. 7 transmits a reading. There can be a short time delay of a few seconds, more or less, before the reading is received. After the reading is received in step 805 the reading is displayed on the user device for inspection by the user. As described above, if the reading is the first reading in a sequence it may be used as an initial reading and shown at the center of a range or indicator. If the user chooses, this reading can be retained as the baseline reading. At step 809 a second reading is received. At step 811, this second reading is compared to the baseline reading. The user device stores the readings in a data register, memory, stack or other storage area for the comparison. At step 813 a change is displayed. The change can be displayed using an indicator arrow, or, as described above, using a bar chart, pie chart, graph, or other display type. After the display is updated the method returns to step 801.

The use of a program such as a smartphone application provides many additional user selections as added features. The user can, for example, change the scale or the sensitivity of the displayed data. The user can set a timed polling interval, if desired, so that the user device automatically interrupts the sensor and obtains a new reading when the time elapses. Alternatively readings can be taken when the user specifically requests a new reading. An alarm or alert can be added for readings that indicate a change in the blood glucose is greater than a threshold. In additional arrangements, various alternative features can be combined in the sensor and displayed with the change in blood glucose, such as pulse, pulse-oximeter, temperature, distance, time and the like to provide on the same screen a variety of biometric information. If these added features are present, the user interface can be configured by the user and various colors, fonts, and styles can be used to tailor the display to the taste of a particular user.

In an aspect of the present application a method for performing an optical non-invasive blood glucose concentration change indication, comprising: providing an optical energy source spaced from a photo-detector by a sensing area; transmitting energy from the optical energy source across human tissue disposed in the sensing area and onto the photo-detector; storing a first reading corresponding to a light intensity observed by the photo-detector; and displaying the first reading on a display of a user device, the first reading corresponding to a baseline blood glucose concentration.

In another aspect of the present application, the above described method is performed and further comprising: subsequently, again transmitting energy from the optical energy source across the tissue disposed in the sensing area and onto the photo-detector; storing a second reading corresponding to the light intensity observed by the photo-detector; determining a difference between the first reading and the second reading; and displaying the difference between the first reading and the second reading indicating a change in blood glucose concentration from the baseline blood glucose concentration.

In yet another aspect of the present application, the above methods are performed and wherein transmitting energy from the optical energy source across human tissue disposed in the sensing area further comprises transmitting energy across a portion of a human ear.

In still another aspect of the present application, the above methods are performed wherein transmitting energy across a portion of the human ear further comprises positioning the optical energy source behind the human ear and positioning the photo-detector adjacent a front portion of the human ear.

In yet another aspect of the present application, the above methods are performed wherein providing an optical energy source spaced from a photo-detector by a sensing area further comprises providing a light emitting diode.

In still another method of the present application, the above methods are performed wherein providing the light emitting diode further comprises providing a red light emitting diode.

In still another method of the present application, the above methods are performed, wherein displaying the first reading on a display of a user device, the first reading corresponding to a baseline blood glucose concentration further comprises transmitting a signal over an over the air interface to the user device.

In yet another alternative aspect of the present application, the above described methods are performed and include wherein transmitting a signal over an over the air interface to the user device further comprises transmitting a signal over a Bluetooth interface.

In yet another aspect of the present application, the above described methods further include receiving the signal at the user device, and displaying an indication on the user device corresponding to the first reading.

In still another aspect of the present application, the above described methods include wherein displaying the difference between the first reading and the second reading indicating a change in blood glucose concentration from the baseline blood glucose concentration further comprises transmitting a signal to a user device using an over the air interface.

In a further aspect of the present application, the above described methods are performed and include receiving the signal using the over the air interface at the user device and displaying a change in blood glucose concentration corresponding to the difference between the first reading and the second reading.

In still another aspect of the present application, the above described methods are performed and further include: prior to transmitting energy from the optical energy source across human tissue disposed in the sensing area and onto the photo-detector, receiving a control signal from a user device from an over the air interface.

In another aspect of the present application, the above described methods are performed wherein the control signal is an interrupt signal.

In still another aspect of the present application, the above described methods are performed and further include displaying the first reading for a user on a display device, the first reading corresponding to a baseline blood glucose concentration further comprises receiving the first reading at a user device over the over the air interface, and receiving an indication from a user that the first reading is a baseline reading.

In still another aspect of the present application, an apparatus includes: an illumination source; a photo-detector spaced from the illumination source by a sensing area configured for insertion of human tissue between the illumination source and the photo-detector; and circuitry coupled to the photo-detector for outputting readings corresponding to light intensity observed by the photo-detector for light transmitted by the illumination source.

In an additional aspect of the present application, the above described apparatus further includes: a transimpedance amplifier coupled to the photo-detector for receiving a current and outputting a voltage corresponding to light received by the photo-detector.

In yet another aspect of the present application, the above described apparatus further includes an analog to digital converter coupled to the transimpedance amplifier and outputting a digital signal corresponding to the voltage.

In still another aspect of the present application, the above described apparatus is provided and further comprises a microcontroller coupled to the analog to digital converter and coupled to the illumination source, and configured to control the illumination source and to store readings corresponding to the digital signal.

In yet another aspect of the present application, the above described apparatus is provided and further comprises a radio transceiver for transmitting data corresponding to the stored readings in the microcontroller.

In an additional aspect of the present application, the above described apparatus is provided and further comprises a user device configured to receive the data transmitted by the radio transceiver. While it is possible to form a user display with various added features, the novel arrangements described herein can be implemented to indicate only a change in blood glucose from a baseline reading. In this arrangement, the indication is solely for blood glucose changes and the claims and the arrangements described do not require these added features.

Although the example embodiments have been described in detail, it should be understood that various changes, substitutions and alterations can be made herein without departing from the spirit and scope of the application as defined by any appended claims.

Moreover, the scope of the present application is not intended to be limited to the particular embodiments of the process, machine, manufacture, and composition of matter, means, methods and steps described in the specification. As one of ordinary skill in the art will readily appreciate from the disclosure, processes, machines, manufacture, compositions of matter, means, methods or steps, presently existing or later to be developed, that perform substantially the same function or achieve substantially the same result as the corresponding embodiments described herein may be utilized according to the embodiments and alternative embodiments. Accordingly, the appended claims are intended to include within their scope such processes, machines, manufacture, compositions of matter, means, methods, or steps.

What is claimed is:

1. A method for performing an optical non-invasive blood glucose concentration change indication, comprising:
providing an optical energy source spaced from a photo-detector by a sensing area;
transmitting energy from the optical energy source across human tissue disposed in the sensing area and onto the photo-detector;
storing a first reading corresponding to a light intensity observed by the photo-detector; and
displaying the first reading on a display of a user device, the first reading corresponding to a baseline blood glucose concentration.

2. The method of claim 1, and further comprising:
subsequently, again transmitting energy from the optical energy source across the tissue disposed in the sensing area and onto the photo-detector;
storing a second reading corresponding to the light intensity observed by the photo-detector;
determining a difference between the first reading and the second reading; and
displaying the difference between the first reading and the second reading indicating a change in blood glucose concentration from the baseline blood glucose concentration.

3. The method of claim 1, wherein transmitting energy from the optical energy source across human tissue disposed in the sensing area further comprises transmitting energy across a portion of a human ear.

4. The method of claim 3, wherein transmitting energy across a portion of the human ear further comprises positioning the optical energy source behind the human ear and positioning the photo-detector adjacent a front portion of the human ear.

5. The method of claim 1, wherein providing an optical energy source spaced from a photo-detector by a sensing area further comprises providing a light emitting diode.

6. The method of claim 5, wherein providing the light emitting diode further comprises providing a red light emitting diode.

7. The method of claim 1, wherein displaying the first reading on a display of a user device, the first reading corresponding to a baseline blood glucose concentration further comprises transmitting a signal over an over the air interface to the user device.

8. The method of claim 7, wherein transmitting a signal over an over the air interface to the user device further comprises transmitting a signal over a Bluetooth interface.

9. The method of claim 7 and further comprising receiving the signal at the user device, and displaying an indication on the user device corresponding to the first reading.

10. The method of claim 2, wherein displaying the difference between the first reading and the second reading indicating a change in blood glucose concentration from the baseline blood glucose concentration further comprises transmitting a signal to a user device using an over the air interface.

11. The method of claim 10 and further comprising receiving the signal using the over the air interface at the user device and displaying a change in blood glucose concentration corresponding to the difference between the first reading and the second reading.

12. The method of claim 1, and further comprising:
prior to transmitting energy from the optical energy source across human tissue disposed in the sensing area and onto the photo-detector, receiving a control signal from a user device from an over the air interface.

13. The method of claim 12, wherein the control signal is an interrupt signal.

14. The method of claim 13, wherein displaying the first reading for a user on a display device, the first reading corresponding to a baseline blood glucose concentration further comprises receiving the first reading at a user device over the over the air interface, and receiving an indication from a user that the first reading is a baseline reading.

15. An apparatus, comprising:
an illumination source;
a photo-detector spaced from the illumination source by a sensing area configured for insertion of human tissue between the illumination source and the photo-detector; and
circuitry coupled to the photo-detector for outputting readings corresponding to light intensity observed by the photo-detector for light transmitted by the illumination source, the readings corresponding to the glucose concentration of blood within the human tissue.

16. The apparatus of claim 15, and further comprising:
a transimpedance amplifier coupled to the photo-detector for receiving a current and outputting a voltage corresponding to light received by the photo-detector.

17. The apparatus of claim 16, and further comprising an analog to digital converter coupled to the transimpedance amplifier and outputting a digital signal corresponding to the voltage.

18. The apparatus of claim 17, and further comprising a microcontroller coupled to the analog to digital converter and coupled to the illumination source, and configured to control the illumination source and to store readings corresponding to the digital signal.

19. The apparatus of claim 18, and further comprising a radio transceiver for transmitting data corresponding to the stored readings in the microcontroller.

20. The apparatus of claim 19, and further comprising a user device configured to receive the data transmitted by the radio transceiver.
